(19) United States
(12) Patent Application Publication
(10) Pub. No.: US 2026/0182886 A1
(43) Pub. Date: Jul. 2, 2026

(54) Boundary Element Method-Based Three-Dimensional Electrophysiology Mapping Method, System and Medium

(71) Applicant: Shenzhen Cardioacc Ltd, Guangdong (CN)

(72) Inventors: Xinhuan Zhou, Guangdong (CN); Jun Ma, Guangdong (CN); Jia Mei, Guangdong (CN)

(21) Appl. No.: 18/727,509

(22) PCT Filed: Sep. 20, 2023
(86) PCT No.: PCT/CN2023/119905

§ 371 Date: Jul. 9, 2024

(30) Foreign Application Priority Data
Apr. 26, 2023 (CN) 202310472347.5

(51) Int. Cl. A61B 5/287; A61B 5/00; A61B 5/346; A61B 8/00; A61B 8/08

(52) U.S. Cl. CPC A61B 5/287; A61B 5/346; A61B 5/743; A61B 8/0883; A61B 8/445; A61B 8/463; A61B 8/466; A61B 8/5223

(57) ABSTRACT

Provided by the present disclosure are a boundary element-based three-dimensional mapping method, system and apparatus, as well as a device and a medium. The method includes the following steps: acquiring a three-dimensional endocardium model, electrode potential data collected by an electrode probe in a cardiac chamber, and position data of the electrode probe collected by a three-dimensional spatial positioning system; performing boundary element discretization processing of the endocardium using a normal vector in geometric vector data, the position data and a preset electrostatic relationship of an intracardiac potential to determine a numerical relationship equation after boundary element discretization; solving an endocardial potential by inverse operation based on the numerical relationship equation, the position data and the electrode potential data; and determining an endocardial charge density on the endocardium according to the endocardial potential data and a preset charge density algorithm, and displaying the endocardial charge density in real time.

### TECHNICAL FIELD

The present disclosure relates to the technical field of electrophysiological mapping, and in particular to a boundary element-based three-dimensional mapping method, system and apparatus, as well as a device, and a medium.

### BACKGROUND

Electrophysiological heart disease (i.e., arrhythmia, including atrial fibrillation, atrial flutter, ventricular flutter, supraventricular tachycardia, etc.) is a major human health problem. There are more than 30 million patients with electrophysiological heart disease in China, and most of them are not well treated, leading to a serious social and economic burden. Electrophysiological heart disease is usually caused by cardiac current conduction system lesion, that is, abnormal conduction sequence and amplitude of current in the heart cause abnormal myocardial contraction.

Catheter interventional ablation is one of the safest and most effective clinical treatments for electrophysiological disease at present. In this surgery, abnormal lesions and electrical signal conduction bundles are ablated by minimally invasive peripheral vein intervention to cut off abnormal current transmission in endocardium or epicardium.

The conduction of catheter interventional ablation needs to map cardiac current transmission first, that is, a catheter tip of the electrode catheter is sent to the hart through femoral venipuncture, and the electrode is used to make contact with multiple different positions on the endocardium or epicardium to record three-dimensionally distributed cardiac exciting currents, and a potential diagram in three-dimensional distribution is used to identify abnormal lesions and conduction bundles such as low voltage areas on the potential diagram, thus determining the target of ablation surgery and guide ablation surgery for treatment. However, it is difficult to achieve a “contact” type mapping operation.

At present, the three-dimensional cardiac mapping techniques used in clinic are all contact type three-dimensional mapping, three-dimensional mapping density of which depends on the number and size of electrodes, so it is necessary to integrate smaller and more electrodes on the electrode catheter to produce high-precision mapping, which is costly. On the other hand, the contact type 3D mapping is time-consuming, but the heartbeat cycle of normal people is 0.8 s, so the contact type 3D mapping needs complicated time and space synchronization, and often needs to assume that the heartbeat is periodic, failure to achieve data support for determining aperiodic arrhythmia.

Therefore, as the existing three-dimensional cardiac mapping technique has the problems of high mapping operation difficulty and inability to support the determination of aperiodic arrhythmia, the existing three-dimensional mapping scheme cannot cover the treatment of complex electrophysiological heart diseases.

### SUMMARY

A main objective of the present disclosure is to provide a boundary element-based three-dimensional mapping method, system and apparatus, as well as a device and a medium, and the problems of high mapping operation difficulty and inability to support the determination of aperiodic arrhythmia in the prior art can be solved.

In order to achieve the objective above, in a first aspect, a boundary element-based three-dimensional mapping method is provided by the present disclosure. The method is applied to a boundary element-based three-dimensional mapping system. The three-dimensional mapping system at least includes a three-dimensional cardiac imaging system, an interventional three-dimensional mapping catheter, a three-dimensional spatial positioning system, and a display system. The interventional three-dimensional mapping catheter includes a multichannel electrode catheter, and a multichannel acquisition system, and multiple mapping electrodes arranged in a certain mode are arranged on the multichannel electrode catheter. The method includes the following steps:
acquiring electrode potential data of the plurality of mapping electrodes collected by the multichannel acquisition system at a current moment, position data of the multichannel electrode catheter collected by the three-dimensional positioning system, and a three-dimensional endocardium model modeled by the three-dimensional cardiac imaging system, where the three-dimensional endocardium model at least includes geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode;
performing boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization;
solving an endocardial potential by inverse operation based on the numerical relationship equation, the position data and the electrode potential data, so as to determine data of the endocardial potentials three-dimensionally distributed on the endocardium; and
determining an endocardial charge density on the endocardium at the current moment according to the endocardial potential data and a preset charge density algorithm, outputting the endocardial charge density to the display system, thus displaying the endocardial charge density on the three-dimensional endocardium model in real time.

In a feasible implementation mode, performing boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization includes the following steps:
traversing the normal vectors in the geometric vector data to obtain a target normal vector with a positive direction being a direction outside the endocardium; when a vector product of a (k+1)^{-th} normal vector and a k-th normal vector is less than 0, flipping a direction of the (k+1)^{-th} normal vector, and enabling k=k+1, and returning to the step that the vector product of the (k+1) th normal vector and the k-th normal vector is less than 0 until the normal vectors of all triangles are traversed, thus obtaining a target normal vector with a positive direction being a direction outside the endocardium, where an initial value of k is 1;
performing boundary element discretization processing based on an endocardial boundary and the position data of the multichannel electrode catheter to determine a transformation matrix between the endocardial potential and the electrode potential after boundary element discretization, where the transformation matrix is used to transform the endocardial potential to a multichannel electrode potential; and
determining a numerical relationship equation between the endocardial potential and the electrode potential after boundary element discretization using the transformation matrix.

In a feasible implementation mode, the numerical relationship equation of the endocardial potential and the electrode potential after boundary element discretization is as follows:

$$A * E 1 = E 2 ;$$

where E1∈R^{M} is endocardial potential data, M represents the number of triangles in the triangular mesh model of the three-dimensional endocardium model, E2∈R^{N} is the electrode potential data collected by the multiple mapping electrodes, N represents the number of mapping electrodes, A∈R^{N*M} is the transformation matrix which is used to transform the endocardial potential to the multichannel electrode potential.

In a feasible implementation mode, the preset electrostatic relationship of the intracardiac potential is as follows:

$$λ ( ε , η , ζ ) E ( ε , η , ζ ) = {\sum }_{k = 1}^{k = M} \{ \overline{E} ( k ) ( ε , η , ζ ) {D}_{2}^{( k )} ( ε , η , ζ ) - {\overline{p}}^{( k )} {D}_{1}^{( k )} ( ε , η , ζ ) \} ;$$

where λ is a target constant coefficient,

$${D}_{1}^{( k )} , {\overline{p}}^{( k )} and {D}_{2}^{( k )}$$

are variables geometrically related to a structure of the boundary, E(ε,η,ζ) represents the endocardial potential and the electrode potential, Ē^{(k)}(ε,η,ζ) is an endocardial potential of a k^{-th} triangle in the intracardiac potential, k∈M.

In a feasible implementation mode, an equation for solving the endocardial potential by inverse operation is as follows:

$$E 1 = {( {A}^{T} * A + λ * I )}^{- 1} * {A}^{T} * E 2 ;$$

where E1∈R^{M} is endocardial potential data, M represents the number of triangles in the triangular mesh model of the three-dimensional endocardium model, E2∈R^{N} is the electrode potential data collected by the multiple mapping electrodes, N represents the number of mapping electrodes, A∈R^{N*M} is a transformation matrix, T represents transposition, λ is a regularization coefficient, I∈R^{M*M} is an unit matrix, where the transformation matrix is used to transform the endocardial potential to the multichannel electrode potential.

In a feasible implementation mode, the charge density algorithm is as follows:

$$E 1 ( \vec{x} ) = {\int }_{s} d ( \vec{y} ) \frac{cos {φ}_{xy}}{{\vert \vec{x} - \vec{y} \vert}^{2}} d {s}_{\vec{y}} ;$$

where \vec{x} and \vec{y} are spatial positions of any point on the boundary of the endocardium, d(\vec{y}) is an endocardial charge density at \vec{y}, φ_{xy} is an included angle between |\vec{x}−\vec{y}| and a normal vector \vec{n}, S is a triangle mesh network, E1(\vec{x}) is an endocardial potential at the spatial position \vec{x} on the boundary of the endocardium.

In order to achieve the objective above, in a second aspect, a boundary element-based three-dimensional mapping system is provided, including three-dimensional cardiac imaging system, an interventional three-dimensional mapping catheter, a three-dimensional spatial positioning system, an ECG (Electrocardiogramaisition system, a display system, and a workstation. The three-dimensional cardiac imaging system, the interventional three-dimensional mapping catheter, the three-dimensional spatial positioning system, the ECG acquisition system and the display system are in communication connection with the workstation, respectively.

The three-dimensional cardiac imaging system is used to perform endocardial modeling by intracardiac echocardiography to obtain a three-dimensional endocardium model, and to upload the three-dimensional endocardium model to the workstation. The three-dimensional endocardium model at least includes geometric vector data of each triangle in a triangular mesh model of the three-dimensional endocardium model.

The interventional three-dimensional mapping catheter includes a multichannel electrode catheter, a multichannel acquisition system, and a control handle. Multiple mapping electrodes arranged in a certain mode are arranged on the multichannel electrode catheter, the mapping electrodes are arranged at a tip of the multichannel electrode catheter, and the control handle is arranged at a distal end of the multichannel electrode catheter far from the tip. The multichannel acquisition system includes multiple data acquisition channels, the data acquisition channels correspond to the mapping electrodes one by one, and the data acquisition channels are used to collect electrode potentials of the mapping electrodes in parallel.

The multiple mapping electrodes form an electrode probe. The electrode probe is inserted into a cardiac chamber in a first shape convenient for intervention, the control handle is used to control the electrode probe to be transformed from the first shape to a second shape convenient for mapping after the probe is inserted into the cardiac chamber. The electrode probe is also used to map electrode potential data in the cardiac chamber in a non-contact manner using the multiple mapping electrodes based on the second shape, and to upload the electrode potential data to the workstation; the electrode potential data is used to reflect blood flow potentials at all parts in the cardiac chamber. The first shape is a closed shape, and the second shape is a non-closed shape.

The three-dimensional spatial positioning system is used to acquire position data of the multichannel electrode catheter and upload the position data to the workstation. The position data is used to reflect a spatial position of the multichannel electrode catheter in the cardiac chamber.

The ECG acquisition system is used to acquire ECG data on a body surface and upload the ECG data to the workstation. The ECG data is used to reflect each heartbeat cycle.

The workstation is used to receive model parameters, the electrode potential data, the position data and the ECG data, to count model parameters, electrode potential data and position data in a current heartbeat cycle based on the ECG data, and to execute the steps of the method in the first aspect and any feasible implementation mode.

The display system is used to display the three-dimensional endocardium model and an endocardial charge density on the three-dimensional endocardium model in real time on a preset display terminal.

In order to achieve the objective above, a boundary element-based three-dimensional mapping apparatus is provided in a third aspect of the present disclosure. The apparatus is applied to a boundary element-based three-dimensional mapping system. The three-dimensional mapping system at least includes a three-dimensional cardiac imaging system, an interventional three-dimensional mapping catheter, a three-dimensional spatial positioning system, and a display system. The interventional three-dimensional mapping catheter includes a multichannel electrode catheter and a multichannel acquisition system, and multiple mapping electrodes arranged in a certain mode are arranged on the multichannel electrode catheter. The apparatus includes:
a cardiac chamber data acquisition module, used to acquire electrode potential data of the multiple mapping electrodes collected by the multichannel acquisition system at a current moment, position data of the multichannel electrode catheter collected by the three-dimensional positioning system, and a three-dimensional endocardium model modeled by the three-dimensional cardiac imaging system, where the three-dimensional endocardium model at least includes geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode;
a potential relationship establishing module, used to perform boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization;
an endocardial potential solution module, used to solve an endocardial potential by inverse operation based on the numerical relationship equation, the position data and the electrode potential data, so as to determine data of the endocardial potentials three-dimensionally distributed on the endocardium; and
a charge density determining module, used to determine an endocardial charge density on the endocardium at the current moment according to the endocardial potential data and a preset charge density algorithm, and to output the endocardial charge density to the display system, thus displaying the endocardial charge density on the three-dimensional endocardium model in real time.

In order to achieve the objective above, a computer readable storage medium is provided in a fourth aspect of the present disclosure. A computer program is stored in the computer readable storage medium. The computer program, when executed by a processor, enables the processor to execute steps of the method in the first aspect and any feasible implementation mode.

In order to achieve the objective above, a computer device is provided in a fifth aspect of the present disclosure, including a memory and a processor. A computer program is stored in the memory. The computer program, when executed by a processor, enables the processor to execute steps of the method in the first aspect and any feasible implementation mode.

The implementation of the embodiment of the present disclosure has the following beneficial effects:

A boundary element-based three-dimensional mapping method is provided by the present disclosure, which is applied to a boundary element-based three-dimensional mapping system. The system at least includes a three-dimensional cardiac imaging system, an interventional three-dimensional mapping catheter, a three-dimensional spatial positioning system, and a display system. The interventional three-dimensional mapping catheter includes a multichannel electrode catheter, and a multichannel acquisition system. Multiple mapping electrodes arranged in a certain mode are arranged on the multichannel electrode catheter. The method includes the following steps: acquiring electrode potential data of the multiple mapping electrodes collected by the multichannel acquisition system at a current moment, position data of the multichannel electrode catheter collected by the three-dimensional positioning system, and a three-dimensional endocardium model modeled by the three-dimensional cardiac imaging system, where the three-dimensional endocardium model at least includes geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode; performing boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization; solving an endocardial potential by inverse operation based on the numerical relationship equation, the position data and the electrode potential data, so as to determine data of the endocardial potentials three-dimensionally distributed on the endocardium; and determining an endocardial charge density on the endocardium at the current moment according to the endocardial potential data and a preset charge density algorithm, outputting the endocardial charge density to the display system, thus displaying the endocardial charge density on the three-dimensional endocardium model in real time.

The electrode potential data collected by the multiple mapping electrodes in the cardiac chamber can reflect the blood flow potential at each position in the cardiac chamber, then through the electrostatic relationship and the transformation matrix, the endocardial potential data can be obtained by using the electrode potential data, and thus the endocardial potential data can be obtained without making contact with the endocardium. As there is no need to make contact with the endocardium, the complexity of mapping operation is reduced, three-dimensional electrophysiological mapping can be performed quickly, and the endocardium three-dimensional mapping can be achieved in one heartbeat cycle. Afterwards, the endocardial charge density in the current heartbeat cycle can be computed through the endocardial potential data, and the endocardial charge density and an endocardium model at the current moment are displayed in real time, which is beneficial to providing data support for determining aperiodic arrhythmia.

### BRIEF DESCRIPTION OF THE DRAWINGS

To describe the technical solutions of the present disclosure or in the prior art more clearly, the following briefly introduces the accompanying drawings required for describing the embodiments or the prior art. Apparently, the accompanying drawings in the following description show merely some embodiments of the present disclosure, and those of ordinary skill in the art may still derive other drawings from these accompanying drawings without creative efforts.

In the drawings:

FIG. 1 is a structural diagram of a boundary element-based three-dimensional mapping system according to an embodiment of the present disclosure;

FIG. 2 is a structural schematic diagram of an interventional three-dimensional mapping catheter according to an embodiment of the present disclosure;

FIG. 3 is a flow chart of a boundary element-based three-dimensional mapping method according to an embodiment of the present disclosure;

FIG. 4 is another flow chart of a boundary element-based three-dimensional mapping method according to an embodiment of the present disclosure;

FIG. 5 is a structural block diagram of a boundary element-based three-dimensional mapping apparatus according to an embodiment of the present disclosure;

FIG. 6 is a structural block diagram of a computer device according to an embodiment of the present disclosure.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the scope of protection of the present disclosure.

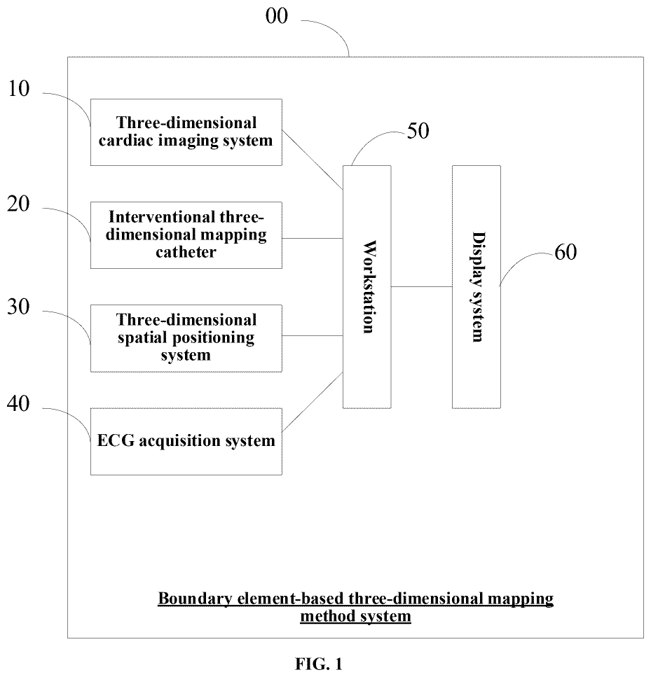

Please referring to FIG. 1, FIG. 1 is a structural diagram of a boundary element-based three-dimensional mapping system 00 according to an embodiment of the present disclosure. The boundary element-based three-dimensional mapping system can be regarded as a non-contact three-dimensional cardiac mapping system. The boundary element-based three-dimensional mapping system 00 shown in FIG. 1 includes a three-dimensional cardiac imaging system 10, an interventional three-dimensional mapping catheter 20, a three-dimensional spatial positioning system 30, an ECG acquisition system 40, a display system 60, and a workstation 50. The three-dimensional cardiac imaging system 10, the interventional three-dimensional mapping catheter 20, the three-dimensional spatial positioning system 30, the ECG acquisition system 40 and the display system 60 are in communication connection with the workstation 50, respectively, and thus can interact data with the workstation 50. In this way, the cardiac excitation current in and His diagram three-dimensional distribution can be recorded, and a user can identify abnormal lesions and conduction bundles such as low voltage areas on a potential diagram through the potential diagram in three-dimensional distribution, so as to give the user guidance to determine the target of ablation surgery and guide ablation surgery for treatment.

Exemplary, the three-dimensional cardiac imaging system 10 includes, but is not limited to, an ultrasonic imaging device capable of achieving endoscopy. The interventional three-dimensional mapping catheter 20 includes, but is not limited to, a multichannel acquisition system with multichannel data acquisition capability, and a multichannel electrode catheter with multichannel data mapping capability. The multichannel electrode catheter is provided with an electrode probe including multiple mapping electrodes arranged in a certain mode, and can be used to intervene in a cardiac chamber to map a blood flow potential in the cardiac chamber based on the multichannel electrode catheter. The three-dimensional spatial positioning system 30 includes, but is not limited to, three-dimensional positioning sensors for magnetic positioning, electrical positioning, or acoustic positioning. The ECG acquisition system 40 includes, but is not limited to, a monitoring apparatus for monitoring an ECG signal on the body surface. The display system 60 includes, but is not limited to, an electronic device with a display screen. The workstation 50 includes, but is not limited to, a terminal or a server, that is, the workstation may include a display system. The terminal may specifically be a desktop terminal, or a mobile terminal, and the mobile terminal may be at least one of a mobile phone, a tablet computer, a notebook computer, and the like. The server may be achieved by an independent server, or a server cluster composed of multiple servers.

It should be noted that the three-dimensional cardiac imaging system 10 is used to perform endocardium modeling by intracardiac echocardiography to obtain model parameters of a three-dimensional endocardium model, and to upload the model parameters to the workstation 50. The model parameters at least include geometric vector data of each triangle in a triangular mesh model of the three-dimensional endocardium model. The geometric vector data includes, but is not limited to, the normal vector of each triangle. Exemplary, intracardiac echocardiography refers to inserting a catheter with an ultrasonic probe into a right cardiac system through peripheral veins to display a cardiac structure image through ultrasound imaging. The three-dimensional endocardium model includes, but is not limited to, a geometric mesh model that approximates an endocardium structure, and the geometric mesh model includes, but is not limited to, a triangular mesh model composed of multiple triangles.

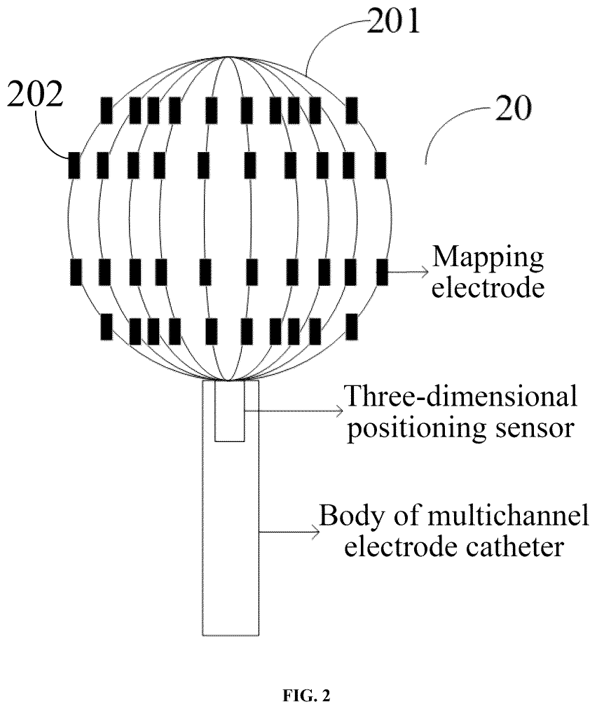

Please referring to FIG. 2, FIG. 2 is a structural schematic diagram of an interventional three-dimensional mapping catheter 20 according to an embodiment of the present disclosure. The interventional three-dimensional mapping catheter 20 includes a multichannel electrode catheter, a multichannel acquisition system, and a control handle. Multiple mapping electrodes 202 arranged in a certain mode are arranged on the multichannel electrode catheter. The mapping electrodes 202 are arranged at a tip of the multichannel electrode catheter, and the control handle is arranged at a distal end of the multichannel electrode catheter far from the tip. The multichannel acquisition system includes multiple data acquisition channels, the data acquisition channels correspond to the mapping electrodes one by one, and the data acquisition channels are used to collect electrode potentials of the mapping electrodes in parallel.

The multiple mapping electrodes form an electrode probe 201. The electrode probe 201 is inserted into a cardiac chamber in a first shape convenient for intervention, the control handle is used to control the electrode probe 201 to be transformed from the first shape to a second shape convenient for mapping after the probe is inserted into the cardiac chamber. The electrode probe 201 is also used to map electrode potential data in the cardiac chamber in a non-contact manner using the multiple mapping electrodes 202 based on the second shape, and to upload the electrode potential data to the workstation 50. The electrode potential data is used to reflect blood flow potentials at all parts in the cardiac chamber, the first shape is a closed shape, and the second shape is a non-closed shape.

The electrode probe 201 includes channel lead wires, and multiple mapping electrodes 202. The mapping electrodes 202 are distributed on the channel lead wires. One end of each channel lead wire is electrically connected to the workstation to upload the electrode potential data collected by the mapping electrode 202 to the workstation, and the other ends of the channel lead wires can be connected to each other to form the first shape when the electrode probe is not inserted into the cardiac chamber, such that the electrode probe can take a shape convenient for intervening in the cardiac chamber; afterwards, when the electrode probe intervenes in the heart, in order to map the blood flow potentials at various positions in the cardiac chamber more conveniently, the other ends of the channel lead wires which are not connected to the workstation can be scattered to maximize the contact between the mapping electrodes and the space in the cardiac chamber, thus mapping the blood flow potentials at various positions in the cardiac chamber.

A non-contact three-dimensional cardiac mapping catheter is shown in FIG. 2. The catheter includes a handle located outside the body, and a bendable catheter that enters the body. The first shape is a closed sphere, and the mapping electrodes 202 (referred to as electrodes) are located on various branches at the tip of the catheter, assuming that a catheter has a total of N electrodes 202. During use, the catheter enters the cardiac chamber through femoral venipuncture, and the handle is used to support a self-expanding branch at the head of the catheter. N electrodes cooperate with an ECG monitoring system on the body surface to record the potentials of blood flow at different electrode positions in the cardiac chamber in one heartbeat cycle. The tip of the catheter is provided with a three-dimensional positioning sensor, which can record a position of the electrode at the tip of the catheter while measuring the blood flow potential. The catheter can enter different cardiac chamber structures. For example, the catheter often needs to puncture the atrial septum to enter the left atrium during atrial fibrillation surgery. It should be noted that the interventional three-dimensional mapping catheter includes, but is not limited to, a three-dimensional cardiac mapping catheter, such as a multichannel electrode catheter with electrode probes, and the above catheter does not need to make contact with the endocardium, thus achieving non-contact three-dimensional cardiac mapping.

The three-dimensional spatial positioning system 30 is used to collect position data of the multichannel electrode catheter and to upload the position data to the workstation 50, and the position data is used to reflect a spatial position of the multichannel electrode catheter in the cardiac chamber. Through the position data, it can be known where the blood flow potential collected by the mapping electrode is in the cardiac chamber. The position data includes, but is not limited to, the position coordinates of the multichannel electrode catheter and the electrode probe and the position coordinates of the mapping electrode. For example, if the distribution position of the mapping electrode on the electrode probe is known, the position coordinates of each mapping electrode can be inferred from the position coordinates of the electrode probe, and then the blood flow potential of a cardiac chamber position corresponding to the position coordinates of each mapping electrode in the cardiac chamber can be obtained.

The ECG acquisition system 40 is used to collect ECG data of the body surface and to upload the ECG data to the workstation 50, and the ECG data is used to reflect each heartbeat cycle. The ECG data includes, but is not limited to, ECG (electrocardiogram), and the heartbeat cycle of the heart can be determined by the ECG data to obtain electrophysiological three-dimensional mapping data of one heartbeat cycle.

The workstation 50 is used to receive model parameters, electrode potential data, position data and ECG data, to count the model parameters, electrode potential data and position data in the current heartbeat cycle based on the ECG data, and to execute the steps of boundary element-based three-dimensional mapping method shown in the present disclosure, so as to achieve the post-processing of the above model parameters, electrode potential data, position data and ECG data and obtain electrophysiological three-dimensional mapping data of one heartbeat cycle. The boundary element-based three-dimensional mapping method may also be called non-contact three-dimensional cardiac mapping method. Further, the display system 60 is used to display the three-dimensional endocardium model and an endocardial charge density on the three-dimensional endocardium model in real time on a preset display terminal. The user can determine or diagnose by observing the endocardial charge density at different moments based on professional knowledge, and guidance is given to the user.

It should be noted that the cardiac electrophysiology is due to the flow and aggregation of potassium ion K⁺, calcium ion Ca²⁺, chloride ion Cl⁻ and other ions in myocardium at different time, the essence of which is the aggregation of charge density in myocardium. The existing three-dimensional mapping technical solutions are all based on the measurement of potential, the potential is the superposition of local and far-field charges. Remote myocardial charge interference will reduce the local measurement results and the potential mapping density. In this case, the accuracy and reliability of three-dimensional mapping can be significantly improved based on the direct measurement of charges.

Exemplary, the non-contact three-dimensional endocardium mapping system is provided in this embodiment. Different imaging sections are imaged in the heart by intracardiac echocardiography (ICE), and the obtained image is subjected to cardiac chamber segmentation. Afterwards, the segmented contour is combined with a three-dimensional position of an imaging probe to obtain three-dimensional surface point cloud, and three-dimensional surface reconstruction is carried out to triangulate the surface of the endocardium.

An ECG gating system is the ECG acquisition system. The body surface ECG acquisition system is used to acquire chest ECG, and to perform time synchronization between the chest ECG and an image collected by intracardiac echocardiography and a signal of multichannel acquisition system.

Catheter is an interventional three-dimensional mapping catheter. The catheter tip includes multiple branches, multiple electrodes are distributed on each branch, the catheter can be propped up by a handle operation, such that the electrodes are distributed in the cardiac chamber, and the blood flow potentials at multiple positions can be collected at the same time. In addition, the catheter tip includes a three-dimensional positioning sensor.

The multichannel acquisition system is as follows. A host is the multichannel acquisition system, each channel communicates with the electrode at a front end of the catheter, and thus multiple channel potentials can be collected and filtered.

The three-dimensional spatial positioning system is a three-dimensional spatial positioning system, i.e., a magnetic field positioning system, an electric field positioning system, and a sound field positioning system, which can record a spatial position and a spatial angle of the three-dimensional positioning sensor at the catheter tip.

An algorithm workstation (i.e., the workstation) is used to perform image segmentation, three-dimensional modeling of endocardial surface, endocardial electrostatic equation discretization by a boundary element method, establish an algebraic equation, and solve potential pseudo-inverse solution. Secondly, a charge density in three-dimensional distribution is calculated based on the measured potential in three-dimensional distribution.

A boundary element method-based three-dimensional cardiac electrophysiological mapping system includes a three-dimensional endocardium modeling system, an ECG gating system, a catheter, a multichannel acquisition system, and a three-dimensional spatial positioning system. The three-dimensional endocardium modeling system can obtain a three-dimensional endocardium model through cardiac imaging, image segmentation, and cloud point three-dimensional reconstruction. The ECG gating system can record an electric signal of the body surface in real time. Finally, the three-dimensional mapping system can transmit the collected filtered signal to the workstation for algorithm post-processing and three-dimensional displaying. A potential acquisition system can acquire a potential of a position where each electrode is located, and perform band-pass filtering to filter high-frequency noise and low-frequency commercial power interference. The three-dimensional positioning system can cooperate with the three-dimensional positioning sensor located at the catheter tip to record a three-dimensional spatial position and a spatial angle of the catheter tip. The acquired filtered multichannel potential signal can be transmitted to the workstation for subsequent processing.

A boundary element-based three-dimensional mapping system is provided. The endocardial potential is obtained by collecting the blood flow potential through the electrode probe of the interventional three-dimensional mapping catheter, which can reduce the number of mapping electrodes and the cost of the catheter. Meanwhile, the purpose of achieving potential mapping on the endocardium can be achieved without making contact with the endocardium, the operation complexity is further reduced, and the sinking of the three-dimensional mapping technique is promoted. Three-dimensional electrophysiological mapping can be performed quickly, and the endocardium three-dimensional mapping and real-time display can be achieved in one heartbeat cycle, thus providing data support for the diagnosis of aperiodic arrhythmia.

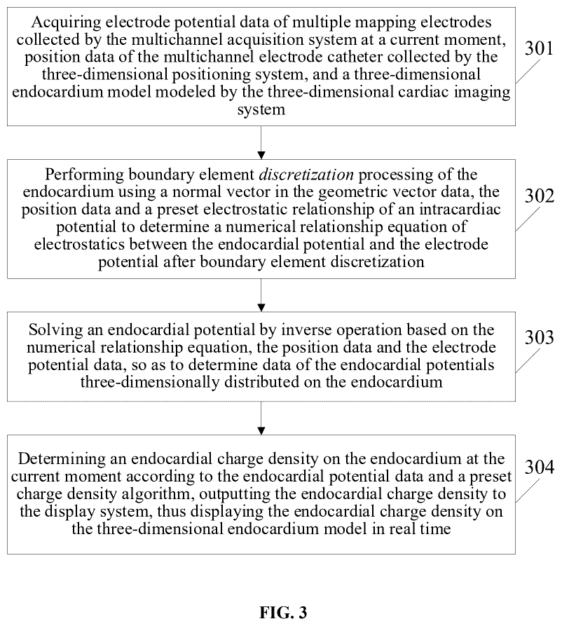

Please referring to FIG. 3, FIG. 3 is a flow chart of a boundary element-based three-dimensional mapping method according to an embodiment of the present disclosure. The boundary element-based three-dimensional mapping method shown in FIG. 3 is applied to a boundary element-based three-dimensional mapping system shown in FIG. 1. The system at least includes a three-dimensional cardiac imaging system, an interventional three-dimensional mapping catheter, a three-dimensional spatial positioning system, and a display system. The interventional three-dimensional mapping catheter includes a multichannel electrode catheter, and a multichannel acquisition system. Multiple mapping electrodes arranged in a certain mode are arranged on the multichannel electrode catheter. The method shown in FIG. 3 includes the following steps.

301. Electrode potential data of the multiple mapping electrodes collected by the multichannel acquisition system at a current moment, position data of the multichannel electrode catheter collected by the three-dimensional positioning system and a three-dimensional endocardium model modeled by the three-dimensional cardiac imaging system are acquired.

The three-dimensional endocardium model at least includes a geometric vector data of each triangle in a triangular mesh model of the three-dimensional endocardium model. The position data is used to reflect a spatial position of the multichannel electrode catheter in a cardiac chamber, and the electrode potential data is used to reflect a blood flow potential at each position in the cardiac chamber corresponding to each mapping electrode.

It should be noted that the related content of the boundary element-based three-dimensional mapping system in this embodiment may refer to the content of the boundary element-based three-dimensional mapping system shown in FIG. 1, which will not be described in detail here for avoiding repetition.

The method shown in this embodiment can be executed by a workstation in the boundary element-based three-dimensional mapping system. The workstation receives the data uploaded by the three-dimensional cardiac imaging system, the interventional three-dimensional mapping catheter, the three-dimensional spatial positioning system and the ECG acquisition system. The current heartbeat cycle can be determined by the ECG acquisition system, and the received data can be synchronized in time based on the current heartbeat cycle. Further, the multichannel electrode potential data collected by the electrode probe in the cardiac chamber and the position data of the multichannel electrode catheter collected by the three-dimensional spatial positioning system are acquired, and the three-dimensional endocardium model is modeled and acquired by the three-dimensional cardiac imaging system to start the three-dimensional electrophysiological mapping in one heartbeat cycle. The geometric data of the three-dimensional endocardium model can be known after obtaining the three-dimensional endocardium model. The three-dimensional endocardium model at least includes geometric vector data of each triangle in the triangular mesh model of the three-dimensional endocardium model, and the position data is used to reflect a spatial position of the multichannel electrode catheter in the cardiac chamber. Because the multichannel electrode catheter intervenes in the cardiac chamber for blood flow potential mapping, the electrode potential data collected by the electrode probe of the multichannel electrode catheter is the blood flow potential at the corresponding spatial position in the cardiac chamber. Therefore, the blood flow potential at each position can be obtained by moving the spatial position of the multichannel electrode catheter in the cardiac chamber, and the electrode potential data is used to reflect the blood flow potential at each position in the cardiac chamber. The geometric data is used to reflect geometric structural parameters and normal parameters of each triangle, which includes, but is not limited to, the normal vector of the triangle.

Exemplary, the three-dimensional endocardium modeling is carried out at first. The three-dimensional endocardium modeling may employ transthoracic ultrasound, esophageal ultrasound and intracardiac echocardiography assisted by the ECG gating system (i.e., ECG acquisition system), or employ imaging devices such as magnetic resonance imaging and CT (Computed tomography) imaging to record a three-dimensional structure of a resting state of the heart (that is, end expiratory and end diastolic). In the present disclosure, the ICE is used, that is, an ultrasonic image and a three-dimensional position of the catheter tip are collected at the end diastolic, and an image obtained by the imaging device is subjected to image segmentation and three-dimensional surface reconstruction by the three-dimensional cardiac imaging system to establish a surface grid of a simply connected domain of the cardiac chamber, which is marked as S. S is a triangle mesh composed of M triangles.

302. Boundary element discretization processing of the endocardium is performed using a normal vector in the geometric vector data, the position data and a preset electrostatic relationship of an intracardiac potential to determine a numerical relationship equation of electrostatics between the endocardial potential and the electrode potential after boundary element discretization.

It should be noted that in order to establish electrostatic numerical computation simulation by a boundary element method, the normal vector of S needs to be computed, and then for each triangular mesh, the normal vector of the k^{-th} triangle of the triangular mesh S can be computed according to its three vertex coordinates:

$${\vec{n}}_{i} = {\vec{a}}_{k} \times {\vec{b}}_{k} \qquad ( 1 )$$

\vec{a}ₖ and \vec{b}ₖ are two edges of the k^{-th} triangular mesh, respectively. In this embodiment, in order to use the boundary element method, a direction pointing out of the endocardium should be defined as a positive direction of the normal vector.

Further, boundary element discretization processing of the endocardium is performed using the normal vector in the geometric vector data, the position data and the preset electrostatic relationship of the intracardiac potential to determine the numerical relationship equation of electrostatics between the endocardial potential and the electrode potential after boundary element discretization.

The intracardiac potential at least includes an endocardial potential and a blood flow potential, which has a certain electrostatic relationship and satisfies Laplace equation. Exemplary, a conduction velocity of a current on the heart is from 0.5 m/s to 7 m/s, which can be approximated as an electrostatic problem, and its control equation is the Laplace equation of the intracardiac potential:

$$Δ E = 0 \qquad ( 2 )$$

Equation (2) is the preset electrostatic relationship of the intracardiac potential, where E is the potential of the blood flow in the endocardium and cardiac chamber.

The boundary element method is used for discretization to obtain the numerical relationship equation of electrostatics between the endocardial potential and the electrode potential after boundary element discretization. Because the electrode potential is used to reflect the blood flow potential, the numerical relationship equation is used to reflect a numerical relationship equation of an electrostatic relationship between the electrode potential everywhere and the endocardial potential, and through the numerical relationship equation, the endocardial potential everywhere can be solved by using the electrode potential everywhere.

303. An endocardial potential is solved by inverse operation based on the numerical relationship equation, the position data and the electrode potential data, so as to determine data of the endocardial potentials three-dimensionally distributed on the endocardium.

Further, the endocardial potential can be solved by inverse operation based on the numerical relationship equation, the position data of the multichannel electrode catheter and the electrode potential data, and the data of endocardial potentials three-dimensionally distributed on the endocardium is determined to obtain the endocardial potential data on the endocardium in the current heartbeat cycle.

304. An endocardial charge density on the endocardium at the current moment is determined according to the endocardial potential data and a preset charge density algorithm, and the endocardial charge density is output to the display system, thus displaying the endocardial charge density on the three-dimensional endocardium model in real time.

Further, the charge distribution on the endocardium at the current moment can be determined through the endocardial potential data. Specifically, the endocardial charge density on the endocardium is determined according to the endocardial potential data and the preset charge density algorithm. The endocardial charge density can be used to reflect the charge distribution on the endocardium. The endocardial charge density is output to the display system, thus displaying the endocardial charge density on the three-dimensional endocardium model. Therefore, a user can evaluate and diagnose according to the displayed content, thus completing endocardial three-dimensional mapping in one heartbeat cycle. The endocardial charge density in one heartbeat cycle is acquired and displayed in real time, which is beneficial to observe the endocardial charge density at different moments and provide data support for the determination of aperiodic arrhythmia.

A boundary element-based three-dimensional mapping method is provided by the present disclosure. The electrode potential data collected by the multiple mapping electrodes in the cardiac chamber can reflect the blood flow potential at each position in the cardiac chamber, and then the endocardial potential data can be obtained using the electrode potential data through the electrostatic relationship and the transformation matrix, thus obtaining the endocardial potential data without making contact with the endocardium. As there is no need to make contact with the endocardium, the complexity of mapping operation is reduced, three-dimensional electrophysiological mapping can be performed quickly, and the endocardium three-dimensional mapping can be achieved in one heartbeat cycle. Afterwards, the endocardial charge density in the current heartbeat cycle can be computed through the endocardial potential data, and the endocardial charge density and an endocardium model at the current moment are displayed in real time, which is beneficial to providing data support for determining aperiodic arrhythmia.

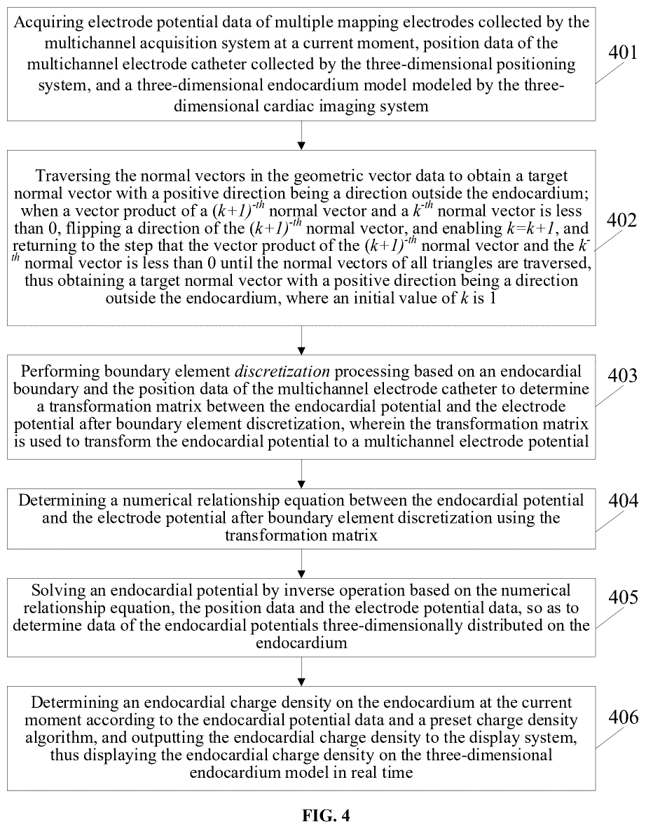

Please referring to FIG. 4, FIG. 4 is another flow chart of a boundary element-based three-dimensional mapping method according to an embodiment of the present disclosure. The boundary element-based three-dimensional mapping method shown in FIG. 4 is applied to the boundary element-based three-dimensional mapping system shown in FIG. 1, and thus will not be described in detail for avoiding repetition. The method shown in FIG. 4 includes the following steps:

401. Electrode potential data of the multiple mapping electrodes collected by the multichannel acquisition system at a current moment, position data of the multichannel electrode catheter collected by the three-dimensional positioning system are acquired, and a three-dimensional endocardium model is modeled and acquired by the three-dimensional cardiac imaging system.

The three-dimensional endocardium model at least includes a geometric vector data of each triangle in a triangular mesh model of the three-dimensional endocardium model. The position data is used to reflect a spatial position of the multichannel electrode catheter in a cardiac chamber, and the electrode potential data is used to reflect a blood flow potential at each position in the cardiac chamber corresponding to each mapping electrode.

It should be noted that the content shown in FIG. 401 is similar to the content of Step 301 shown in FIG. 3, and thus will not be described in detail here for avoiding repetition, specifically referring to the content of Step 301 of the method shown in FIG. 3.

402. The normal vectors in the geometric vector data are traversed to obtain a target normal vector with a positive direction being a direction outside the endocardium. When a vector product of a (k+1)^{-th} normal vector and a k^{-th} normal vector is less than 0, a direction of the (k+1)^{-th} normal vector is flipped, and k is enabled to be equal to k+1, and it is returned to execute the step that the vector product of the (k+1)^{-th} normal vector and the k^{-th} normal vector is less than 0 until the normal vectors of all triangles are traversed, thus determining a target normal vector with a positive direction being the direction outside the endocardium. An initial value of k is 1.

Further, the normal vector of each triangle mesh is computed according to the three-dimensional endocardium model, and all normal vectors are traversed to determine that a positive direction of the normal vector is a direction pointing out of the endocardium. Because the normal vector of the plane has two directions, in order to improve the accuracy of the boundary element method, the normal vectors of each triangle need to be unified in the same direction, the same direction is a specified positive direction of the normal vector, where the specified positive direction is a direction pointing out of the endocardium. Therefore, after obtaining the normal vector of each triangle, the directions can be determined one by one for direction unification. Specifically, the normal vectors in the geometric vector data are traversed to obtain the target normal vector with the positive direction being the direction outside the endocardium. When the vector product of the (k+1)^{-th} normal vector and the k^{-th} normal vector is less than 0, the direction of the (k+1)^{-th} normal vector is flipped, and k=k+1, and it is returned to execute the step that the vector product of the (k+1)^{-th} normal vector and the k^{-th} normal vector is less than 0 until the normal vectors of all triangles are traversed, thus obtaining target normal vector with the positive direction being the direction outside the endocardium. An initial value of the k is 1.

Exemplary, for each triangular mesh, the normal vector of the k^{-th} triangle of the triangular mesh S can be calculated according to its three vertex coordinates:

$${\vec{n}}_{i} = {\vec{a}}_{k} \times {\vec{b}}_{k}$$

\vec{a}ₖ and \vec{b}ₖ are two edges of the k^{-th} triangular mesh, respectively. In this embodiment, in order to use the boundary element method, the direction pointing out of the endocardium should be defined as the positive direction of the normal vector. Therefore, the M triangles in the triangular mesh S need to be traversed, if \vec{n}ₖ·\vec{n}ₖ₊₁<0, \vec{n}ₖ₊₁=\vec{n}ₖ₊₁. A result of cross multiplication between two normal vectors is less than 0, indicating that the included angle between the two normal vectors is 180 degrees, that is, the directions of the two normal vectors are opposite. Then, the direction of the (k+1)^{-th} normal vector is flipped to be the same as the k^{-th} normal vector. The normal vectors of each triangle k are traversed one by one to get the normal vectors in the same direction, that is, the positive direction is a target normal vector pointing to the direction outside the endocardium.

403. Boundary element discretization processing is performed based on an endocardial boundary and the position data of the multichannel electrode catheter to determine a transformation matrix between the endocardial potential and the electrode potential after boundary element discretization. The transformation matrix is used to transform the endocardial potential to a multichannel electrode potential.

The endocardium and the electrode catheter are subjected to boundary element discretization to determine the transformation matrix from endocardial potential to the electrode potential on electrode catheter. The transformation matrix includes constant coefficients, where the rules for taking the constant coefficients include constant coefficients corresponding to endocardial potentials inside, outside and on the boundaries of the endocardium, and the endocardial potentials at least include the endocardial potential and the blood flow potential. Specifically, after obtaining the endocardial boundary, the constant coefficients of the intracardiac potentials with different positions from the boundary can be determined. For example, the intracardiac potential inside the boundary corresponds to a first constant coefficient, the intracardiac potential outside the boundary correspond to a second constant coefficient, and the intracardiac potential on the boundary corresponds to a third constant coefficient. The first constant coefficient may be 0.5, the second constant coefficient can be 0, and the third constant coefficient can be 1. The target constant coefficient of the intracardiac potential is obtained through the above constant coefficient rules. The transformation matrix after boundary discretization can be obtained, and the endocardial potential can be obtained by performing inverse operation based on the transformation matrix and the electrode potential. That is, the transformation matrix is used to transform the potentials at multiple triangular mesh points on the three-dimensional endocardium model to the potentials on the multichannel electrode.

404. The numerical relationship equation between the endocardial potential and the electrode potential after boundary element discretization is determined using the transformation matrix.

Further, the electrostatic relationship satisfying Laplace equation is discretized by the boundary element method, and then the numerical relationship equation between the endocardial potential and the electrode potential after boundary element discretization is determined.

Exemplary, the conduction velocity of current on the heart is from 0.5 m/s to 7 m/s, which can be approximated as an electrostatic problem, and its control equation is the Laplace equation of the intracardiac potential:

$$Δ E = 0 \qquad ( 2 )$$

Equation (2) is the preset electrostatic relationship of the intracardiac potential, where E is the potential of blood flow in the endocardium and the cardiac chamber. The method for establishing the boundary element method is as follows:

$$λ ( ε , η , ζ ) E ( ε , η , ζ ) = {\sum }_{k = 1}^{k = M} \{ \overline{E} ( k ) ( ε , η , ζ ) {D}_{2}^{( k )} ( ε , η , ζ ) - {\overline{p}}^{( k )} {D}_{1}^{( k )} ( ε , η , ζ ) \} \qquad ( 3 )$$

λ in Equation (3) is a target constant coefficient, for example, for the potential on the boundary, λ=0.5; for the potential outside the boundary, λ=0; and for the potential inside the boundary, λ=1. D₁^{(k)}, p̅^{(k)} and D₂^{(k)} are some variables geometrically related to a cardiac wall, the cardiac wall may be understood as the boundary of the endocardium, and will not be described more in the present disclosure. E(ε,η,ζ) represents the potentials of the endocardium and the electrode, Ē^{(k)}(ε,η,ζ) is an endocardial potential of a k^{-th} triangle in the intracardiac potential, k=M.

When E(ε,η,ζ) and all are the potentials located on the boundary, Ē^{(k)} can be computed using simultaneous equations. E(ε,η,ζ) is taken as a potential at any point inside the cardiac chamber (potential at the electrode position), Ē^{(k)}(ε,η,ζ) is the potential located on the boundary, and thus an algebraic equation after discretization can be obtained as follows:

$$A * E 1 = E 2 \qquad ( 4 )$$

where Equation (4) can be regarded as a numerical relationship equation of the electrostatics of the intracardiac potential after discretization. The equation is an equation group obtained by combining the blood flow potentials collected by N mapping electrodes E2, E1∈R^{M} is the endocardial potential, i.e., endocardial potential data. M represents the number of triangles of a triangular mesh model of the three-dimensional endocardium model. E2∈R^{N} is the potentials on N electrodes, i.e., the electrode potential data collected by the electrode probe, N represents the number of the mapping electrodes on the electrode probe, A∈R^{N*M} is the transformation matrix, and the transformation matrix is used to transform the endocardial potential to the multichannel electrode potential.

405. An endocardial potential is solved by inverse operation based on the numerical relationship equation, the position data and the electrode potential data, so as to determine data of the endocardial potentials three-dimensionally distributed on the endocardium.

It should be noted that the content shown in Step 405 is similar to the content of Step 303 shown in FIG. 3, and thus will not be described in detail here for avoiding repetition, specifically referring to the content of Step 303 shown in FIG. 3.

Exemplary, the potential on the electrode is collected. The electrode catheter is inserted into the cardiac chamber to make contact with the blood flow. The potentials on the multiple electrodes in one heartbeat cycle are collected, and the collected signals are filtered and denoised. Meanwhile, a three-dimensional positioning sensor is used to record a spatial position of each electrode relative to the triangular mesh S. The recorded electrode potential is E1, the electrode potential is the blood flow potential. A potential signal collected by the multichannel electrode catheter is filtered to establish an inverse matrix from the electrode potential to the endocardial potential, and then the endocardial potential is solved by inverse operation based on the numerical relationship equation, the position data and the electrode potential data. Equation (4) above is an ill-conditioned matrix, and then the ill-conditioned equation is solved. Because the solution of the ill-conditioned matrix is not unique and particularly sensitive to an input error, in order to enhance the numerical stability of the system, the present disclosure proposes to use Tikhonov Regularization method, i.e., a solving Equation (5):

$$( {A}^{T} * A + λ * I ) * E 1 = {A}^{T} * E 2 \qquad ( 5 )$$

λ in Equation (5) is a regularization coefficient, which is obtained according to experience. I∈R^{M*N} is a unit matrix. The equation (5) may be solved using multiple methods, e.g., Gauss-Siedel iteration, GMRES (generalized minimal residual), CG (conjugate gradient), and other methods.

$$E 1 = {( {A}^{T} * A + λ * I )}^{- 1} * {A}^{T} * E 2 \qquad ( 6 )$$

Equation (6) can be regarded as a solution equation for inverse operation of the endocardial potential. According to Equation (6), the endocardial potential can be solved by the inverse operation, and E1 can be computed after collecting E2. That is, the endocardial potential is computed by the blood flow potential in the cardiac chamber measured on the electrode catheter. In the equation, E1∈R^{M} is endocardial potential data, M represents the number of triangles in the triangular mesh model of the three-dimensional endocardium model, E2∈R^{N} is the electrode potential data collected by the electrode potential, N represents the number of mapping electrodes on the electrode probe to collect blood flow potentials at different spatial positions, A∈R^{N*M} is the transformation matrix, T represents transposition, λ*I is a unit matrix. The transformation matrix is used to transform the endocardial potential to the multichannel electrode potential. It may be understood that the electrode potential data includes electrode potentials at different spatial positions, each electrode potential represents the blood flow potential at different spatial positions, and the endocardial potential data includes endocardial potential at each position of the endocardium.

406. An endocardial charge density on the endocardium at the current moment is determined according to the endocardial potential data and a preset charge density algorithm, and the endocardial charge density is output to the display system, thus displaying the endocardial charge density on the three-dimensional endocardium model in real time.

It should be noted that the content shown in Step 406 is similar to the content of Step 304 shown in FIG. 3, and thus will not be described in detail here for avoiding repetition, specifically referring to the content of Step 304 shown in FIG. 3.

Exemplary, the charge density algorithm is as follows:

$$E 1 ( \vec{x} ) = {\int }_{s} d ( \vec{y} ) \frac{cos {φ}_{xy}}{{\vert \vec{x} - \vec{y} \vert}^{2}} {ds}_{\vec{y}} ; \qquad ( 7 )$$

Equation (7) is a charge density algorithm, in the equation, \vec{x} and \vec{y} are spatial positions of any point on the boundary of the endocardium, d(\vec{y}) is the endocardial charge density at \vec{y}, φ_{xy} is an included between |\vec{x}−\vec{y}| and the normal vector \vec{n}, S is a triangular mesh network, E1(\vec{x}) is an endocardial potential at the spatial position \vec{x} on the boundary of the endocardium.

According to boundary element-based three-dimensional mapping method provided by the present disclosure, the electrode potential data collected by the multiple mapping electrodes in the cardiac chamber can reflect a blood flow potential at each position in the cardiac chamber. Then, the endocardial potential data is obtained using the electrode potential data through the transformation matrix, thus achieving potential transformation, and obtaining the endocardial potential data without making contact with the endocardium. As there is no need to make contact with the endocardium, the complexity of mapping operation is reduced, three-dimensional electrophysiological mapping speed is improved, and the endocardium three-dimensional mapping can be achieved in one heartbeat cycle. Moreover, the endocardial charge density in the current heartbeat cycle can be computed through the endocardial potential data, and the endocardial charge density and the endocardium model at the current moment are displayed in real time, which is beneficial to providing data support for determining aperiodic arrhythmia.

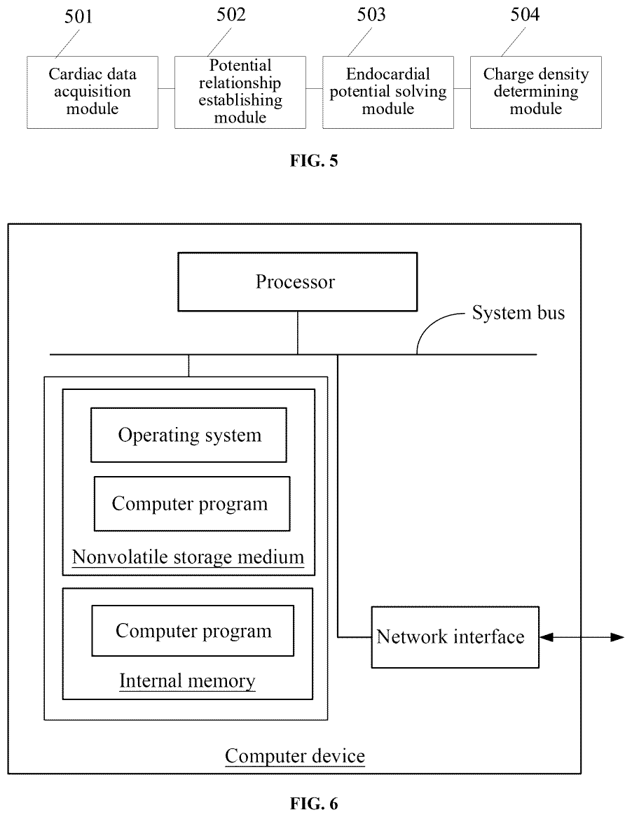

Please referring to FIG. 5, FIG. 5 is a structural block diagram of a boundary element-based three-dimensional mapping apparatus. The apparatus shown in FIG. 5 is applied to a boundary element-based three-dimensional mapping system. The three-dimensional mapping system at least includes a three-dimensional cardiac imaging system, an interventional three-dimensional mapping catheter, a three-dimensional spatial positioning system, and a display system. The interventional three-dimensional mapping catheter includes a multichannel electrode catheter and a multichannel acquisition system. Multiple mapping electrodes arranged in a certain mode are arranged on the multichannel electrode catheter. The apparatus includes:
a cardiac chamber data acquisition module 501, used to acquire electrode potential data of the plurality of mapping electrodes collected by the multichannel acquisition system at a current moment, position data of the multichannel electrode catheter collected by the three-dimensional positioning system, and a three-dimensional endocardium model modeled by the three-dimensional cardiac imaging system, where the three-dimensional endocardium model at least comprises geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode;
a potential relationship establishing module 502, used to perform boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization;
an endocardial potential solution module 503, used to solve an endocardial potential by inverse operation based on the numerical relationship equation, the position data and the electrode potential data, so as to determine data of the endocardial potentials three-dimensionally distributed on the endocardium; and
a charge density determining module 504, used to determine an endocardial charge density on the endocardium at the current moment according to the endocardial potential data and a preset charge density algorithm, and to output the endocardial charge density to the display system, thus displaying the endocardial charge density on the three-dimensional endocardium model in real time.

It should be noted that the function of each module in the apparatus shown in FIG. 5 is similar to the content of each step of the method shown in FIG. 3, and thus will not be described in detail here for avoiding repetition, specifically referring to the steps in the method shown in FIG. 3.

A boundary element-based three-dimensional mapping apparatus is provided by the present disclosure, which is applied to a boundary element-based three-dimensional mapping system. The boundary element-based three-dimensional mapping system at least includes a three-dimensional cardiac imaging system, an interventional three-dimensional mapping catheter, a three-dimensional spatial positioning system, and a display system. The interventional three-dimensional mapping catheter includes a multichannel electrode catheter, and a multichannel acquisition system. Multiple mapping electrodes arranged in a certain mode are arranged on the multichannel electrode catheter. The apparatus includes: a cardiac chamber data acquisition module, used to acquire electrode potential data of the plurality of mapping electrodes collected by the multichannel acquisition system at a current moment, position data of the multichannel electrode catheter collected by the three-dimensional positioning system, and a three-dimensional endocardium model modeled by the three-dimensional cardiac imaging system, where the three-dimensional endocardium model at least comprises geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode; a potential relationship establishing module, used to perform boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization; an endocardial potential solution module, used to solve an endocardial potential by inverse operation based on the numerical relationship equation, the position data and the electrode potential data, so as to determine data of the endocardial potentials three-dimensionally distributed on the endocardium; and a charge density determining module, used to determine an endocardial charge density on the endocardium at the current moment according to the endocardial potential data and a preset charge density algorithm, and to output the endocardial charge density to the display system, thus displaying the endocardial charge density on the three-dimensional endocardium model in real time. The electrode potential data collected by the multiple mapping electrodes in the cardiac chamber can reflect the blood flow potential at each position in the cardiac chamber, and then the endocardial potential data can be obtained using the electrode potential data through the electrostatic relationship and the transformation matrix, thus obtaining the endocardial potential data without making contact with the endocardium. As there is no need to make contact with the endocardium, the complexity of mapping operation is reduced, three-dimensional electrophysiological mapping speed is improved, and the endocardium three-dimensional mapping can be achieved in one heartbeat cycle. Moreover, the endocardial charge density in the current heartbeat cycle can be computed through the endocardial potential data, and the endocardial charge density and the endocardium model at the current moment are displayed in real time, which is beneficial to providing data support for determining aperiodic arrhythmia.

FIG. 6 is a diagram of an internal structure of a computer device according to an embodiment. The computer device may specifically be a terminal, or a server. As shown in FIG. 6, the computer device includes a processor, a memory and a network interface which are connected by a system bus. The memory includes a non-volatile storage medium, and an internal memory. The non-volatile storage medium of the computer device stores an operating system, and can also store a computer program. The computer program, when executed by the processor, can make the processor implement the above method. The computer program may also be stored in the internal memory. The computer program, when executed by a processor, enables the processor to execute the above method. Those skilled in the part may understand that the structure shown in FIG. 6 is only a block diagram of a part of the structure related to the scheme of the present disclosure, and does not constitute a limitation on the computer device to which the scheme of the present disclosure is applied. The specific computer device may include more or less components than those shown in the figure, or combine some components, or have different component arrangements.

In one embodiment, a computer device is further disclosed, including a memory, and a processor. A computer program is stored in the memory, and the computer program, when executed by the processor, enables the processor to execute the steps in the method shown in FIG. 3 or FIG. 4.

In one embodiment, a computer readable storage medium is further disclosed, and a computer program is stored in the computer readable storage medium. The computer program, when executed by a processor, enables the processor to execute the steps in the method shown in FIG. 3 or FIG. 4.

Those skilled in the art can understand that all or part of the processes in the method for implementing the above embodiments can be completed by instructing related hardware through the computer program, which can be stored in a nonvolatile computer-readable storage medium, and the program, when executed, may include the processes of the above embodiments. Any reference to memory, storage, database or other media used in the embodiments provided in the present disclosure may include non-volatile and/or volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM), or an external cache memory. By way of illustration than limitation, RAM is available in various forms, such as static RAM (SRAM), dynamic RAM (DRAM), synchronous DRAM (SDRAM), double data rate SDRAM (DDRSDRAM), enhanced SDRAM (ESDRAM), synchlink DRAM (SLDRAM), memory bus (Rambus) direct RAM (RDRAM), and direct rambus dynamic RAM (DRDRAM), and rambus dynamic RAM (RDRAM).

The technical features of the above embodiments can be combined at will. In order to make the description concise, not all possible combinations of the technical features in the above embodiments are described. However, it should be considered that these combinations of technical features fall within the scope recorded in this specification provided that these combinations of technical features do not have any conflict.

The foregoing embodiments only describe several implementation modes of the present disclosure, and their description is specific and detailed, but cannot therefore be understood as a limitation to the patent scope of the present disclosure. It should be noted that for those of ordinary skill in the art, several deformations and improvements can be made without departing from the concept of the present disclosure, all of which fall within the scope of protection of the present disclosure. Therefore, the scope of protection of the patent of the present disclosure shall be subject to the appended claims.

1. A boundary element-based three-dimensional mapping method, wherein the method is applied to a boundary element-based three-dimensional mapping system, the three-dimensional mapping system at least comprises a three-dimensional cardiac imaging system, an interventional three-dimensional mapping catheter, a three-dimensional spatial positioning system, and a display system; the interventional three-dimensional mapping catheter comprises a multichannel electrode catheter, and a multichannel acquisition system, and a plurality of mapping arranged in a certain mode are arranged on the multichannel electrode catheter, and the method comprises the following steps:
acquiring electrode potential data of the plurality of mapping electrodes collected by the multichannel acquisition system at a current moment, position data of the multichannel electrode catheter collected by the three-dimensional positioning system, and a three-dimensional endocardium model modeled by the three-dimensional cardiac imaging system, wherein the three-dimensional endocardium model at least comprises geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode;
performing boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization;
solving an endocardial potential by inverse operation based on the numerical relationship equation, the position data and the electrode potential data, so as to determine data of the endocardial potentials three-dimensionally distributed on the endocardium; and
determining an endocardial charge density on the endocardium at the current moment according to the endocardial potential data and a preset charge density algorithm, outputting the endocardial charge density to the display system, thus displaying the endocardial charge density on the three-dimensional endocardium model in real time.

2. The method according to claim 1, wherein performing boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization comprises the following steps:
traversing the normal vectors in the geometric vector data to obtain a target normal vector with a positive direction being a direction outside the endocardium; when a vector product of a (k+1)^{-th} normal vector and a k^{-th} normal vector is less than 0, flipping a direction of the (k+1)^{-th} normal vector, and enabling k=k+1, and returning to the step that the vector product of the (k+1)^{-th} normal vector and the k^{-th} normal vector is less than 0 until the normal vectors of all triangles are traversed, thus obtaining a target normal vector with a positive direction being a direction outside the endocardium, wherein an initial value of k is 1;
performing boundary element discretization processing based on an endocardial boundary and the position data of the multichannel electrode catheter to determine a transformation matrix between the endocardial potential and the electrode potential after boundary element discretization, wherein the transformation matrix is used to transform the endocardial potential to a multichannel electrode potential; and
determining a numerical relationship equation between the endocardial potential and the electrode potential after boundary element discretization using the transformation matrix.

3. The method according to claim 1, wherein the numerical relationship equation between the endocardial potential and the electrode potential after boundary element discretization is as follows:
$$A * E 1 = E 2 ;$$
wherein E1∈R^{M} is endocardial potential data, M represents the number of triangles in the triangular mesh model of the three-dimensional endocardium model, E2∈R^{N} is the electrode potential data collected by the plurality of mapping electrodes, N represents the number of mapping electrodes, A∈R^{N*M} is the transformation matrix which is used to transform the endocardial potential to the multichannel electrode potential.

4. The method according to claim 1, wherein the preset electrostatic relationship of the intracardiac potential is as follows:
$$λ ( ε , η , ζ ) E ( ε , η , ζ ) = {\sum }_{k = 1}^{k = M} \{ \overline{E} ( k ) ( ε , η , ζ ) {D}_{2}^{( k )} ( ε , η , ζ ) - {\overline{p}}^{( k )} {D}_{1}^{( k )} ( ε , η , ζ ) \} ;$$
wherein λ is a target constant coefficient,
$${D}_{1}^{( k )} , {\overline{p}}^{( k )} and {D}_{2}^{( k )}$$
are variables geometrically related to a structure of the boundary, E(ε,η,ζ) represents the endocardial potential and the electrode potential, Ē^{(k)}(ε,η,ζ) is an endocardial potential of a k^{-th} triangle in the intracardiac potential, k∈M.

5. The method according to claim 1, wherein an equation for solving the endocardial potential by inverse operation is as follows:
$$E 1 = {( {A}^{T} * A + λ * I )}^{- 1} * {A}^{T} * E 2 ;$$
wherein E1∈R^{M} is endocardial potential data, M represents the number of triangles in the triangular mesh model of the three-dimensional endocardium model, E2∈R^{N} is the electrode potential data collected by the plurality of mapping electrodes, N represents the number of mapping electrodes, A∈R^{N*M} is a transformation matrix, T represents transposition, λ is a regularization coefficient, I∈R^{M*M} is an unit matrix, wherein the transformation matrix is used to transform the endocardial potential to the multichannel electrode potential.

6. The method according to claim 1, wherein the charge density algorithm is as follows:
$$E 1 ( \vec{x} ) = {\int }_{s} d ( \vec{y} ) \frac{cos {φ}_{xy}}{{\vert \vec{x} - \vec{y} \vert}^{2}} {ds}_{\vec{y}} ;$$
wherein \vec{x} and \vec{y} are spatial positions of any point on the boundary of the endocardium, d(\vec{y}) is an endocardial charge density at \vec{y}, φ_{xy} is an included angle between |\vec{x}−\vec{y}| and a normal vector \vec{n}, s is a triangle mesh network, E1(\vec{x}) is an endocardial potential at the spatial position \vec{x} on the boundary of the endocardium.

7. A boundary element-based three-dimensional mapping system, comprising a three-dimensional cardiac imaging system, an interventional three-dimensional mapping catheter, a three-dimensional spatial positioning system, an ECG (Electrocardiogram system, a display system, and a workstation; wherein the three-dimensional cardiac imaging system, the interventional three-dimensional mapping catheter, the three-dimensional spatial positioning system, the ECG acquisition system and the display system are in communication connection with the workstation, respectively;
the three-dimensional cardiac imaging system is used to perform endocardial modeling by intracardiac echocardiography to obtain a three-dimensional endocardium model, and upload the three-dimensional endocardium model to the workstation, wherein the three-dimensional endocardium model at least comprises geometric vector data of each triangle in a triangular mesh model of the three-dimensional endocardium model;
the interventional three-dimensional mapping catheter comprises a multichannel electrode catheter, a multichannel acquisition system, and a control handle, wherein a plurality of mapping electrodes arranged in a certain mode are arranged on the multichannel electrode catheter, the mapping electrodes are arranged at a tip of the multichannel electrode catheter, and the control handle is arranged at a distal end of the multichannel electrode catheter far from the tip; the multichannel acquisition system comprises a plurality of data acquisition channels, the data acquisition channels correspond to the mapping electrodes one by one, and the data acquisition channels are used to collect electrode potentials of the mapping electrodes in parallel;
the plurality of mapping electrodes form an electrode probe, the electrode probe is inserted into a cardiac chamber in a first shape convenient for intervention, the control handle is used to control the electrode probe to be transformed from the first shape to a second shape convenient for mapping after the probe is inserted into the cardiac chamber, and the electrode probe is also used to map electrode potential data in the cardiac chamber in a non-contact manner using the plurality of mapping electrodes based on the second shape, and to upload the electrode potential data to the workstation; the electrode potential data is used to reflect blood flow potentials at all parts in the cardiac chamber, the first shape is a closed shape, and the second shape is a non-closed shape;
the three-dimensional spatial positioning system is used to acquire position data of the multichannel electrode catheter and upload the position data to the workstation, wherein the position data is used to reflect a spatial position of the multichannel electrode catheter in the cardiac chamber;
the ECG acquisition system is used to acquire ECG data on a body surface and upload the ECG data to the workstation, wherein the ECG data is used to reflect each heartbeat cycle;
the workstation is used to receive model parameters, the electrode potential data, the position data and the ECG data, to count model parameters, electrode potential data and position data in a current heartbeat cycle based on the ECG data, and to execute the steps of the method according to claim 1; and
the display system is used to display the three-dimensional endocardium model and an endocardial charge density on the three-dimensional endocardium model in real time on a preset display terminal.

8. A boundary element-based three-dimensional mapping apparatus, wherein the apparatus is applied to a boundary element-based three-dimensional mapping system, the three-dimensional mapping system at least comprises a three-dimensional cardiac imaging system, an interventional three-dimensional mapping catheter, a three-dimensional spatial positioning system, and a display system; the interventional three-dimensional mapping catheter comprises a multichannel electrode catheter and a multichannel acquisition system, and a plurality of mapping electrodes arranged in a certain mode are arranged on the multichannel electrode catheter, and the apparatus comprises:
a cardiac chamber data acquisition module, used to acquire electrode potential data of the plurality of mapping electrodes collected by the multichannel acquisition system at a current moment, position data of the multichannel electrode catheter collected by the three-dimensional positioning system, and a three-dimensional endocardium model modeled by the three-dimensional cardiac imaging system, wherein the three-dimensional endocardium model at least comprises geometric vector data of each triangle in a triangular mesh model of the three-dimensional endocardium model, the position data is used to reflect a spatial position of the multichannel electrode catheter in a cardiac chamber, and the electrode potential data is used to reflect a blood flow potential at each position in the cardiac chamber corresponding to each mapping electrode;
a potential relationship establishing module, used to perform boundary element discretization processing of the endocardium using a normal vector in the geometric vector data, the position data and a preset electrostatic relationship of an intracardiac potential to determine a numerical relationship equation of electrostatics between the endocardial potential and the electrode potential after boundary element discretization;
an endocardial potential solution module, used to solve an endocardial potential by inverse operation based on the numerical relationship equation, the position data and the electrode potential data, so as to determine data of the endocardial potentials three-dimensionally distributed on the endocardium; and
a charge density determining module, used to determine an endocardial charge density on the endocardium at the current moment according to the endocardial potential data and a preset charge density algorithm, and to output the endocardial charge density to the display system, thus displaying the endocardial charge density on the three-dimensional endocardium model in real time.

9. A computer readable storage medium, wherein a computer program is stored in the computer readable storage medium, and the computer program, when executed by a processor, enables the processor to execute the steps of the method according to claim 1 to 6.

10. A computer device, comprising a memory and a processor, wherein a computer program is stored in the memory, and the computer program, when executed by the processor, enables the processor to execute the steps of the method according to claim 1.
